(19) United States
(12) Patent Application Publication
(10) Pub. No.: US 2023/0060040 A1
(43) Pub. Date: Feb. 23, 2023

(54) USE OF TRANSFERRIN, TRANSFERRIN RECEPTOR AND ANTIBODY THEREOF IN PREPARATION OF ANTI-SARS-CoV-2 DRUG

(71) Applicants: KUNMING INSTITUTE OF ZOOLOGY, CHINESE ACADEMY OF SCIENCES, Yunnan (CN); INSTITUTE OF MEDICAL BIOLOGY, CHINESE ACADEMY OF MEDICAL SCIENCES, Yunnan (CN)

(72) Inventors: Ren LAI, Kunming (CN); Xiaozhong PENG, Kunming (CN); Xiaopeng TANG, Kunming (CN); Zhiyi LIAO, Kunming (CN); Shuaiyao LU, Kunming (CN); Hongqi LIU, Kunming (CN)

(21) Appl. No.: 17/415,782

(22) PCT Filed: Jun. 29, 2020
(86) PCT No.: PCT/CN2020/098861

§ 371 Date: Jun. 18, 2021

(51) Int. Cl. A61K 38/40; A61P 31/14; A61K 38/17; A61K 38/02

(52) U.S. Cl. CPC A61K 38/40; A61P 31/14; A61K 38/177; A61K 38/02

(57) ABSTRACT

The present disclosure provides use of transferrin, a transferrin receptor and an antibody thereof in the preparation of an anti-SARS-CoV-2 drug. Use of the transferrin, the transferrin receptor or the transferrin receptor antibody in the preparation of an anti-SARS-CoV-2 drug is provided. Both surface plasmon resonance (SPR) and immunofluorescence confirm that the SARS-CoV-2 binds to the transferrin receptor through SARS-CoV-2 spike protein; the transferrin and/or the transferrin receptor antibody competitively bind(s) to the transferrin receptor of the body, or the transferrin receptor competitively binds to a site of the SARS-CoV-2 to inhibit binding of the SARS-CoV-2 to the transferrin receptor of the body; thus, the opportunity that the SARS-CoV-2 infects cells is blocked, and the antiviral effect of the body is realized. Use of the transferrin, the transferrin receptor and the antibody thereof in the preparation of a SARS-CoV-2 spike protein-binding biological product is provided

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Aug. 15, 2022, is named 32422-002-Sequence-Listing.txt and is 932 bytes in size.

### TECHNICAL FIELD

The present disclosure belongs to the technical field of antiviral drugs, and specifically relates to use of transferrin, a transferrin receptor and an antibody thereof in the preparation of an anti-SARS-CoV-2 drug.

### BACKGROUND ART

Since the 21th century, severe acute respiratory syndrome coronavirus (SARS-CoV), Middle East respiratory syndrome coronavirus (MERS-CoV), and severe acute respiratory syndrome coronavirus 2 (SARS-CoV-2) have crossed the species barrier and been transmitted to humans through animals, causing human to suffer from severe pneumonia. The outbreak of SARS-CoV has been contained successfully through public health intervention measures such as early detection and quarantine. The SARS-CoV-2 was discovered in December, 2019 and sequenced and isolated in January, 2020. On Jan. 30, 2020, the World Health Organization (WHO) declared that the outbreak of SARS-CoV-2 constituted a public health emergency of international concern (PHEIC) [1-3]. The SARS-CoV-2 is an enveloped virus with a positive RNA genome and belongs to the subfamily Coronavirinae. There are four genera (α,β,γ, and δ), and β-CoV is further divided into four species (A, B, C, and D).

Transferrin is a main iron-containing protein in plasma. The molecular weight of transferrin is about 77,000. As a single-chain glycoprotein, transferrin is responsible for transporting iron absorbed by the digestive tract and released by red blood cell degradation. The research on physiological functions of transferrin at the present stage has shown that in addition to the function of transporting iron ions, transferrin also has important functions such as antibiosis and participation in cell growth and differentiation[4]. Transferrin further delivers iron to cells through the endocytic pathway of the transferrin receptor, so the transferrin receptor plays an important role in cellular iron homeostasis[5].

[1] A. C. Walls et al., Structure, Function, and Antigenicity of the SARS-CoV-2 Spike Glycoprotein. Cell 181, 281-292 e286 (2020).

[2] R. Li et al., Substantial undocumented infection facilitates the rapid dissemination of novel coronavirus (SARS-CoV-2). Science 368, 489-493 (2020).

[3] W. Wang et al., Detection of SARS-CoV-2 in Different Types of Clinical Specimens. JAMA, (2020).

[4] P. T. Gomme, K. B. McCann, J. Bertolini, Transferrin: structure, function and potential therapeutic actions. Drug Discov Today 10, 267-273 (2005).

[5] H. Fuchs, U. Lucken, R. Tauber, A. Engel, R. Gessner, Structural model of phospholipid-reconstituted human transferrin receptor derived by electron microscopy. Structure 6, 1235-1243 (1998).

### SUMMARY

In view of this, an objective of the present disclosure is to provide new use of transferrin, that is, use of transferrin in the preparation of an anti-SARS-CoV-2 drug.

Another objective of the present disclosure is to provide use of a transferrin receptor or a transferrin receptor antibody in the preparation of an anti-SARS-CoV-2 drug.

The present disclosure provides use of transferrin in the preparation of an anti-SARS-CoV-2 drug.

Preferably, the transferrin may have a concentration of not less than 250 nmol/L.

The present disclosure provides use of a transferrin receptor in defense against SARS-CoV-2.

The present disclosure provides use of a transferrin receptor in the preparation of an anti-SARS-CoV-2 drug.

Preferably, the transferrin receptor may have a concentration of not less than 100 nmol/L.

The present disclosure provides use of a transferrin receptor in defense against SARS-CoV-2.

The present disclosure provides use of a transferrin receptor antibody in the preparation of an anti-SARS-CoV-2 drug.

Preferably, the transferrin receptor antibody may have a concentration of not less than 70 nmol/L.

The present disclosure provides use of a transferrin receptor antibody in defense against SARS-CoV-2.

The present disclosure provides use of transferrin and/or a transferrin receptor antibody in the preparation of a SARS-CoV-2 spike protein-binding biological product.

The present disclosure provides a SARS-CoV-2 spike protein-binding biological product, including transferrin and/or a transferrin receptor antibody.

The present disclosure provides use of a transferrin receptor in the preparation of a SARS-CoV-2 spike protein-binding biological product.

The present disclosure provides an anti-SARS-CoV-2 composition, including transferrin and a transferrin receptor antibody; the transferrin and the transferrin receptor antibody may have a molar ratio of not less than 1:1.

The present disclosure provides use of the composition in the preparation of an anti-SARS-CoV-2 drug.

The present disclosure provides an anti-SARS-CoV-2 drug, including active ingredients and excipients, where the active ingredients include transferrin and a transferrin receptor antibody;

the transferrin and the transferrin receptor antibody have a molar ratio of not less than 1:1.

The present disclosure provides the use of transferrin in the preparation of an anti-SARS-CoV-2 drug. The present disclosure uses two methods, surface plasmon resonance (SPR) and immunofluorescence, to confirm that SARS-CoV-2 binds to the transferrin receptor through spike protein at the cellular level. Therefore, the transferrin is used to competitively bind to the transferrin receptor of the body, the active site of the transferrin receptor is blocked and the binding of the SARS-CoV-2 to the transferrin receptor is inhibited, thereby blocking the pathway of the SARS-CoV-2 infecting cells and achieving the antiviral effect of the body. According to experiments, using remdesivir as a positive control drug, treatment of SARS-CoV-2-infected cells with transferrin concludes that the transferrin can effectively inhibit SARS-CoV-2 infection, and the inhibitory potency is dose-dependent.

The present disclosure provides the use of a transferrin receptor in the preparation of an anti-SARS-CoV-2 drug. The present disclosure uses two methods, SPR and immunofluorescence, to confirm that SARS-CoV-2 binds to the transferrin receptor through SARS-CoV-2 spike protein at the cellular level. Therefore, the transferrin receptor is used to competitively bind to the transferrin receptor of the body, the active site of the transferrin receptor is blocked and the binding of the SARS-CoV-2 to the transferrin receptor in the body is inhibited, thereby blocking the pathway of the SARS-CoV-2 infecting cells and achieving the antiviral effect of the body. According to experiments, using remdesivir as a positive control drug, treatment of SARS-CoV-2-infected cells with transferrin receptor concludes that the transferrin receptor can effectively inhibit SARS-CoV-2 infection, and the inhibitory potency is dose-dependent.

The present disclosure provides the use of a transferrin receptor antibody in the preparation of an anti-SARS-CoV-2 drug. The present disclosure uses two methods, SPR and immunofluorescence, to confirm that SARS-CoV-2 binds to the transferrin receptor through SARS-CoV-2 spike protein at the cellular level. Therefore, the transferrin receptor antibody is used to competitively bind to the transferrin receptor, the active site of the transferrin receptor is blocked and the binding of the SARS-CoV-2 to the transferrin receptor in the body is inhibited, thereby blocking the pathway of the SARS-CoV-2 infecting cells and achieving the antiviral effect of the body. According to experiments, using remdesivir as a positive control drug, treatment of SARS-CoV-2-infected cells with transferrin receptor monoclonal antibody concludes that the transferrin receptor monoclonal antibody can effectively inhibit SARS-CoV-2 infection, and the inhibitory potency is dose-dependent.

### BRIEF DESCRIPTION OF THE DRAWINGS

FIG. 1 illustrates the verification of the binding of SARS-CoV-2-spike (spike protein) to transferrin receptor by SPR and immunofluorescence in Examples 1 and 2; where Panel A shows the verification of the binding of SARS-CoV-2-spike (spike protein) to transferrin receptor by SPR, where the curve, from top to bottom, represents 250 nM, 125 nM, 62.5 nM, 31.25 nM, 15.625 nM, 7.8125 nM, and 3.90625 nM; Panel B illustrates the result of the verification of the binding of SARS-CoV-2-spike (spike protein) to transferrin receptor by immunofluorescence;

FIG. 2 illustrates the morphology of SARS-CoV-2-infected Vero E6 cells treated with positive control drug remdesivir in Comparative Example 1;

FIG. 3 illustrates a comparison of different concentrations (31.25 nM, 62.5 nM, 125 nM, 250 nM, 500 nM, and 1,000 nM) of transferrin in the treatment of SARS-CoV-2-infected Vero E6 cells in Example 3;

FIG. 4 illustrates the statistical results of the inhibition rate of transferrin against SARS-CoV-2-infected cells by CPE and qPCR in Example 3, where Panel A shows the statistical results of the CPE analysis of the inhibition rate of transferrin against SARS-CoV-2-infected cells; Panel B shows the statistical results of the qPCR analysis of the inhibition rate of transferrin against SARS-CoV-2 infected cells;

FIG. 5 illustrates a comparison of different concentrations (25 nM, 50 nM, 100 nM, 200 nM, 400 nM, and 800 nM) of transferrin receptor in the treatment of SARS-CoV-2-infected Vero E6 cells in Example 4;

FIG. 6 illustrates the statistical results of the inhibition rate of transferrin receptor against SARS-CoV-2-infected cells by CPE and qPCR in Example 4, where Panel A shows the statistical results of the CPE analysis of the inhibition rate of transferrin against SARS-CoV-2-infected cells; Panel B shows the statistical results of the qPCR analysis of the inhibition rate of transferrin against SARS-CoV-2 infected cells;

FIG. 7 illustrates a comparison of different concentrations (12.5 nM, 25 nM, 50 nM, 100 nM, 200 nM, and 400 nM) of transferrin receptor monoclonal antibody in the treatment of SARS-CoV-2-infected Vero E6 cells in Example 5;

FIG. 8 illustrates the statistical results of the inhibition rate of transferrin receptor monoclonal antibody against SARS-CoV-2-infected cells by CPE and qPCR in Example 5, where Panel A shows the statistical results of the CPE analysis of the inhibition rate of transferrin against SARS-CoV-2-infected cells; Panel B shows the statistical results of the qPCR analysis of the inhibition rate of transferrin against SARS-CoV-2 infected cells.

### DETAILED DESCRIPTION OF THE EMBODIMENTS

The present disclosure provides use of transferrin in the preparation of an anti-SARS-CoV-2 drug.

Sources of the transferrin are not specifically limited in the present disclosure, as long as the transferrin well known in the art may be used. In the examples of the present disclosure, the transferrin is purchased from Sigma, with a catalog number of T4382. Serial gradient concentrations of the transferrin are used to treat SARS-CoV-2-infected cells. The results show that the transferrin may effectively inhibit SARS-CoV-2 infection, and the inhibitory potency is dose-dependent. Cytopathogenic effect (CPE) analysis results show that the transferrin has an EC₅₀ of 125 nmol/L; quantitative analysis of reverse transcription real-time quantitative PCR (qRT-PCR) shows that the transferrin has an EC₅₀ of 160 nmol/L. The transferrin may preferably have a concentration of not less than 250 nmol/L.

The present disclosure provides use of a transferrin receptor in the preparation of an anti-SARS-CoV-2 drug.

Sources of the transferrin receptor are not specifically limited in the present disclosure, as long as the transferrin receptor well known in the art may be used. In the examples of the present disclosure, the transferrin receptor is purchased from Sino Biological, with a catalog number of 11020-H07H. Serial gradient concentrations of the transferrin receptor are used to treat SARS-CoV-2-infected cells. The results show that the transferrin receptor may effectively inhibit SARS-CoV-2 infection, and the inhibitory potency is dose-dependent. CPE analysis results show that the transferrin receptor has an EC₅₀ of 80 nmol/L; quantitative analysis of reverse transcription qRT-PCR shows that the transferrin receptor has an EC₅₀ of 93 nmol/L. The transferrin receptor may have a concentration of not less than 100 nmol/L.

The present disclosure provides use of a transferrin receptor antibody in the preparation of an anti-SARS-CoV-2 drug.

Categories of the transferrin receptor antibody are not specifically limited in the present disclosure, as long as either polyclonal or monoclonal antibody well known in the art may be used. Sources of the antibody are not specifically limited in the present disclosure, as long as the transferrin receptor antibody well known in the art may be used. In the examples of the present disclosure, the transferrin receptor monoclonal antibody is purchased from Abcam, with a catalog number of ab1086. Serial gradient concentrations of the transferrin receptor monoclonal antibody are used to treat SARS-CoV-2-infected cells. The results show that the transferrin receptor monoclonal antibody may effectively inhibit SARS-CoV-2 infection, and the inhibitory potency is dose-dependent. CPE analysis results show that the transferrin receptor monoclonal antibody has an EC₅₀ of 80 nmol/L; quantitative analysis of reverse transcription qRT-PCR shows that the transferrin receptor has an EC₅₀ of 50 nmol/L. The transferrin receptor may have a concentration of not less than 16.6 nmol/L. The transferrin receptor antibody may have a concentration of not less than 70 nmol/L.

Experiments have demonstrated that the transferrin or the transferrin receptor antibody exerts antiviral effect by blocking the binding of SARS-CoV-2 to the transferrin receptor in the body, and binding thereto is achieved by binding a spike protein of the virus to the transferrin receptor in the body. Therefore, the present disclosure provides use of transferrin and/or a transferrin receptor antibody in the preparation of a SARS-CoV-2 spike protein-binding biological product. Meanwhile, the present disclosure further provides use of a transferrin receptor in the preparation of a SARS-CoV-2 spike protein-binding biological product. Categories of the biological product are not specifically limited in the present disclosure, as long as the biological product well known in the art may be prepared.

The present disclosure provides an anti-SARS-CoV-2 composition, including transferrin and a transferrin receptor antibody; the transferrin and the transferrin receptor antibody may have a molar ratio of not less than 1:1.

Sources of the transferrin and the transferrin receptor antibody are not specifically limited in the present disclosure, as long as the transferrin and the transferrin receptor antibody well known in the art may be used. The transferrin and the transferrin receptor antibody may preferably have a molar ratio of (2-5):1, and more preferably (3-4):1.

The present disclosure provides use of the composition in the preparation of an anti-SARS-CoV-2 drug.

Dosage forms of the drug are not specifically limited in the present disclosure, as long as the dosage forms well known in the art may be used. The drug may include different types of excipients according to different dosage forms. The types of excipients are not specifically limited in the present disclosure, as long as the excipients well known in the art may be used. Preparation methods of the drug are not specifically limited in the present disclosure, as long as the preparation methods of the drug well known in the art may be used.

The present disclosure provides use of the transferrin, a transferrin receptor or a transferrin receptor antibody in defense against SARS-CoV-2. Sources of the transferrin, the transferrin receptor, and the transferrin receptor antibody are not specifically limited in the present disclosure, as long as the sources of the transferrin, the transferrin receptor, and the transferrin receptor antibody well known in the art may be used.

The present disclosure provides an anti-SARS-CoV-2 drug, including active ingredients and excipients, and the active ingredients include transferrin and a transferrin receptor antibody; the transferrin and the transferrin receptor antibody may have a molar ratio of not less than 1:1, preferably (2-5):1, and more preferably (3-4):1. The content and types of the excipients are not specially limited in the present disclosure, as long as the excipients well known in the art may be used. Preparation methods of the drug are not specifically limited in the present disclosure, as long as the preparation methods of the drug well known in the art may be used.

The use of transferrin, a transferrin receptor and an antibody thereof in the preparation of an anti-SARS-CoV-2 drug provided by the present disclosure will be described in detail below in conjunction with examples, but should not be construed as limiting the protection scope of the present disclosure.

### Example 1

Verification of the binding of SARS-CoV spike protein to transferrin receptor by SPR

BIAcore 2000 (General Electric Company, USA) was used to analyze the interaction between transferrin receptor and spike protein. The transferrin receptor (Cat. No. 11020-H07H) was diluted to 20 μg/ml with 200 μl of sodium acetate buffer (10 mM, pH 5), and flew through a CMS sensor chip (BR100012, GE) at a flow rate of 5 μl/min to reach 2,000 resonance units (RU); the remaining active sites on the chip were blocked with 75 μl of ethanolamine solution (1 M, pH 8.5). The interactions between serial concentrations of spike proteins (3.90625 nM, 7.8125 nM, 15.625 nM, 31.25 nM, 62.5 nM, 125 nM, 250 nM; catalog number Z03481) and immobilized transferrin receptor were analyzed at a flow rate of 10 μl/min. The BIA software (GE, USA) was used to determine the bound KD and the Ka and Kd rate constants.

The results are shown in FIG. 1A. From FIG. 1A, the SARS-CoV-2 spike protein binds to the transferrin receptor with strong binding capacity.

### Example 2

Verification of the binding of SARS-CoV-2-spike protein to transferrin receptor at the cellular level by immunofluorescence

To detect the transferrin receptor and spike protein complexes on the membrane surface of Vero E6 cells, the cells (MOCK) were infected with SARS-CoV-2 (MOI=0.2) for 2 h, and uninfected MOCK was used as a control. After washing with PBS, the MOCK cells were fixed with 4% paraformaldehyde in PBS for 15 min, blocked with 1% BSA solution at room temperature for 1 h, and then incubated together with anti-transferrin receptor antibody (1:200 dilution; 11020-MMO4, Sino Biological, China) and anti-spike protein (1:200 dilution; 40150-R007, Sino Biological, China) antibody at 37° C. for 1 h. After washing thrice with PBS to remove excess primary antibody, sections were incubated with fluorescently labeled secondary antibody at 37° C. for 1 h. After washing with PBS to remove excess secondary antibody, the cells were stained with DAPI (P36941, Life Technologies, USA) and imaged with a confocal microscope (FluoView™1000, Olympus, USA).

The results are shown in FIG. 1B. The SARS-CoV-2 spike protein binds to the transferrin receptor at the cellular level.

### Comparative Example 1

Remdesivir was used as a positive control drug to investigate the effect thereof on SARS-CoV-2-infected African green monkey embryonic kidney cells (Vero E6).

Specific steps were as follows: Vero E6 cells were pretreated with remdesivir (4 μM) for 1 h, and infected with SARS-CoV-2 virus for 1 h. Subsequently, the virus-protein mixture was removed, and the cells were further cultured in a fresh medium supplemented with 4 μM remdesivir. After 48 h treatment, a cell supernatant was collected and lysed with lysis buffer (15596018, Thermo, USA). The morphology of the cells before and after treatment was observed and photographed under a microscope.

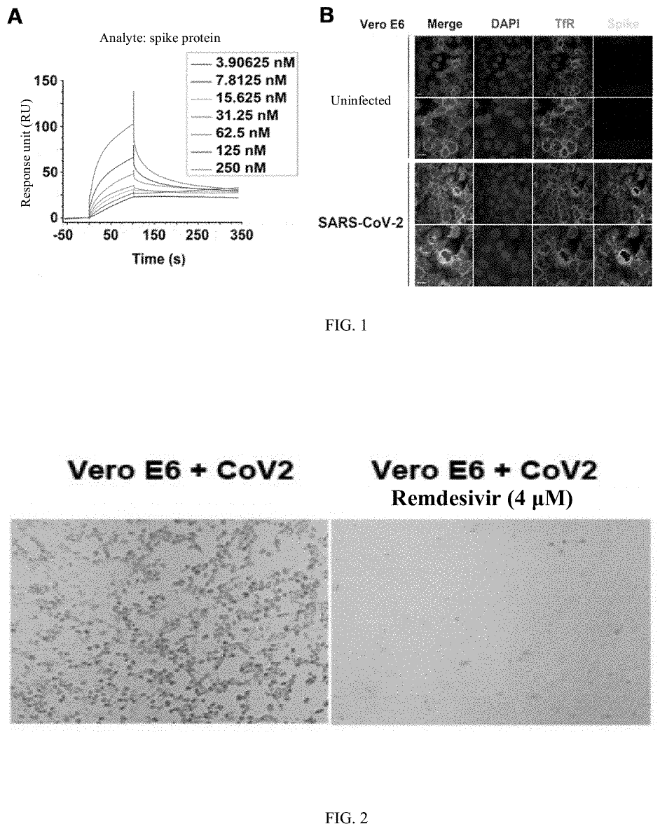

The results are shown in FIG. 2. The comparison of the morphology of the cells before and after treatment revealed that remdesivir could inhibit SARS-CoV-2 to infect cells.

### Example 3

SARS-CoV-2-infected Vero E6 cells were treated with different concentrations of transferrin.

The Vero E6 cells were pretreated with different concentrations (31.25 nM, 62.5 nM, 125 nM, 250 nM, 500 nM, and 1,000 nM) of transferrin for 1 h, and infected with SARS-CoV-2 for 1 h. Subsequently, the virus-protein mixture was removed, and the cells were further cultured in fresh media supplemented with different concentrations of transferrin (31.25 nM, 62.5 nM, 125 nM, 250 nM, 500 nM, and 1,000 nM). After culturing for 48 h, a cell supernatant was collected and lysed with lysis buffer (15596018, Thermo, USA). The morphology of the cells before and after treatment was observed and photographed under a microscope.

Subsequently, CPE (refer to M. Wang et al., Remdesivir and chloroquine effectively inhibit the recently emerged novel coronavirus (2019-nCoV) in vitro. Cell Res 30, 269-271 (2020)) and quantitative RT-PCR (qRT-PCR) were used for quantitative analysis. Herein, the RT-qPCR was operated in accordance with the instructions, and RNA extraction kit (DP419) and reverse transcription kit (A5000) were used for RNA extraction and cDNA reverse transcription program, respectively. The detection method of RT-qPCR was as follows:

Nucleotide sequences of NP gene primers and a probe were as follows.

Target-2-F: 5′-ggggaacttctcctgctagaat-3′ (SEQ ID No. 1);

Target-2-R: 5′-cagacallllgctctcaagctg-3′ (SEQ ID No. 2); and

Target-2-P: 5′-FAM-ttgctgctgcttgacagatt-TAMRA-3′ (SEQ ID No. 3).

The PCR program was as follows: 25° C. for 2 min; 40 cycles of 50° C. for 2 min, 95° C. for 2 min, 95° C. for 5 s, and 58° C. for 31 s. The PCR system was as follows: 0.5 μl each of forward primer F, reverse primer and fluorescent probe P; 2.5 μl of 4×qPCR MIX,; finally, and ddH₂O to make up to 10 μl.

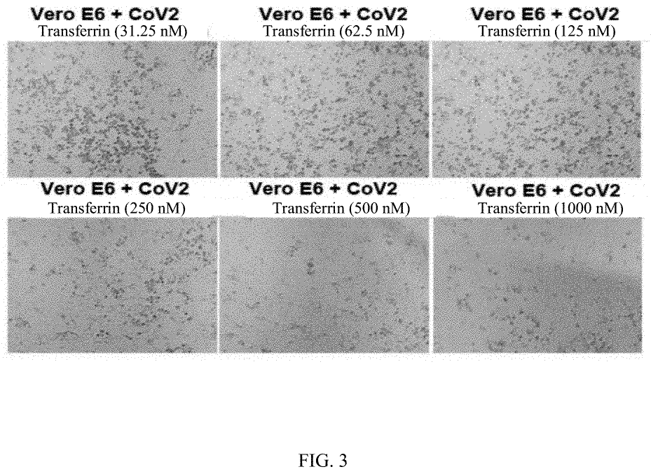

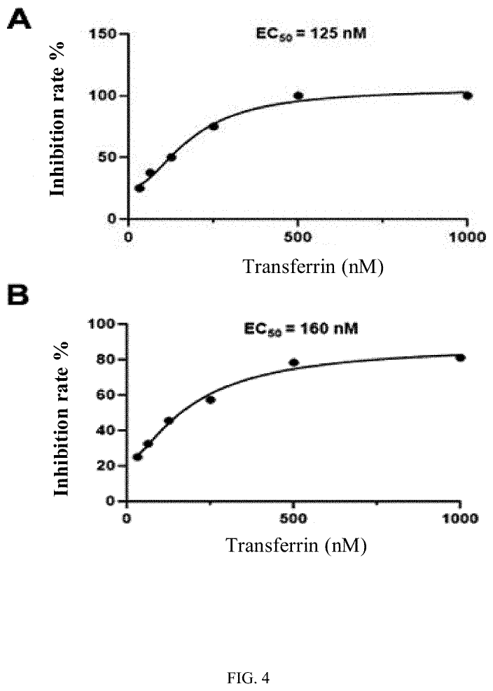

The results are shown in FIGS. 3 and 4. According to the above results, the EC₅₀ of transferrin calculated by CPE was 125 nM, and the EC₅₀ of transferrin calculated by RT-qPCR was 160 nM. As the transferrin concentration increased, the transferrin had a strong inhibitory effect on SARS-CoV-2. Transferrin could inhibit SARS-CoV-2 infection.

### Example 4

SARS-CoV-2-infected Vero E6 cells were treated with different concentrations (25 nM, 50 nM, 100 nM, 200 nM, 400 nM, and 800 nM) of transferrin receptor. Refer to the description in Example 3 for specific steps.

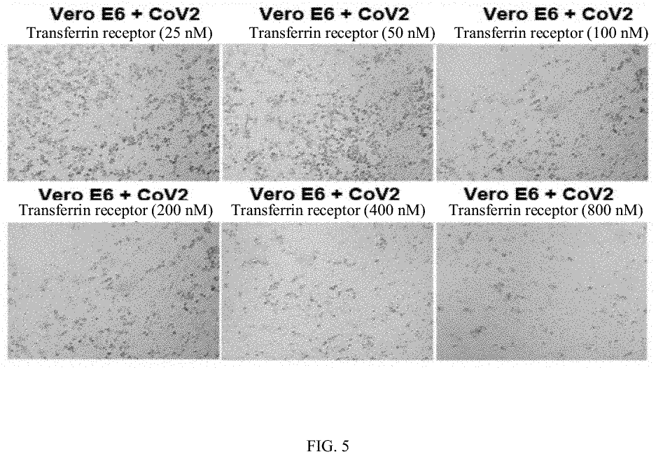

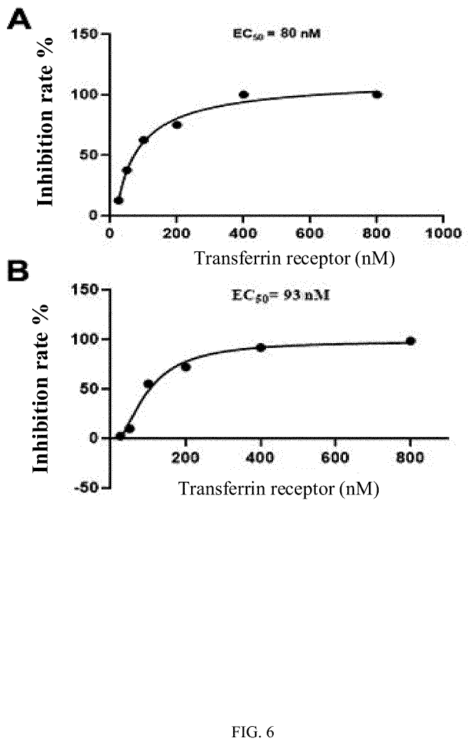

The results are shown in FIGS. 5 and 6. According to the above results, the EC₅₀ of transferrin calculated by CPE was 80 nM, and the EC₅₀ of transferrin calculated by RT-qPCR was 93 nM. As the transferrin concentration increased, the transferrin had a strong inhibitory effect on SARS-CoV-2. The transferrin receptor could inhibit SARS-CoV-2 infection.

### Example 5

SARS-CoV-2-infected Vero E6 cells were treated with different concentrations of transferrin receptor monoclonal antibody. Refer to the description in Example 3 for specific steps.

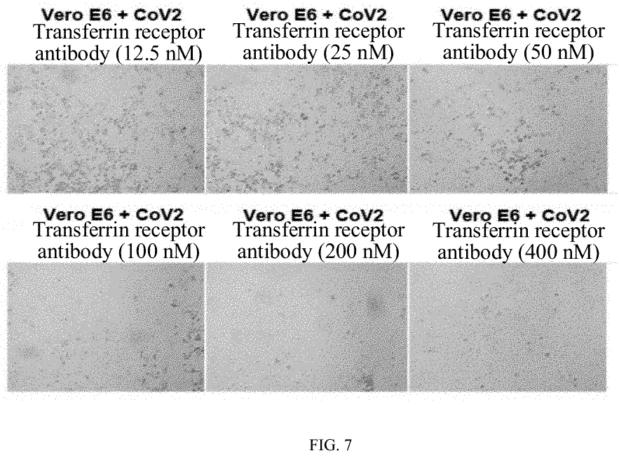

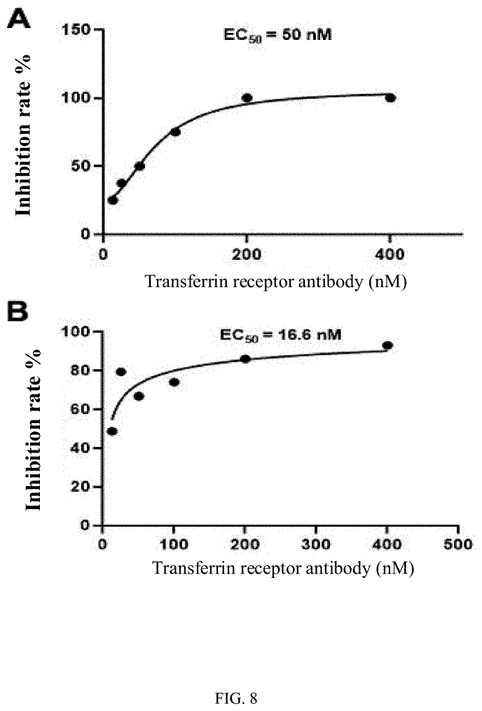

The results are shown in FIGS. 7 and 8. According to the above results, the EC₅₀ of transferrin calculated by CPE was 50 nM, and the EC₅₀ of transferrin calculated by RT-qPCR was 16.6 nM. As the transferrin concentration increased, the transferrin had a strong inhibitory effect on SARS-CoV-2. The transferrin receptor monoclonal antibody could inhibit SARS-CoV-2 infection.

The above descriptions are merely preferred implementations of the present disclosure. It should be noted that a person of ordinary skill in the art may further make several improvements and modifications without departing from the principle of the present disclosure, and such improvements and modifications should be deemed as falling within the protection scope of the present disclosure.

What is claimed is:

1. Use of transferrin in the preparation of an anti-SARS-CoV-2 drug.

2. The use according to claim 1, wherein the transferrin has a concentration of not less than 250 nmol/L.

3. Use of a transferrin receptor in defense against SARS-CoV-2.

4. Use of a transferrin receptor in the preparation of an anti-SARS-CoV-2 drug.

5. The use according to claim 4, wherein the transferrin receptor has a concentration of not less than 100 nmol/L.

6. Use of a transferrin receptor in defense against SARS-CoV-2.

7. Use of a transferrin receptor antibody in the preparation of an anti-SARS-CoV-2 drug.

8. The use according to claim 7, wherein the transferrin receptor antibody has a concentration of not less than 70 nmol/L.

9. Use of a transferrin receptor antibody in defense against SARS-CoV-2.

10. Use of transferrin and/or a transferrin receptor antibody in the preparation of a SARS-CoV-2 spike protein-binding biological product.

11. A SARS-CoV-2 spike protein-binding biological product, comprising transferrin and/or a transferrin receptor antibody.

12. Use of a transferrin receptor in the preparation of a SARS-CoV-2 spike protein-binding biological product.

13. An anti-SARS-CoV-2 composition, comprising transferrin and a transferrin receptor antibody; wherein the transferrin and the transferrin receptor antibody have a molar ratio of not less than 1:1.

14. Use of the composition according to claim 13 in the preparation of an anti-SARS-CoV-2 drug.

15. An anti-SARS-CoV-2 drug, comprising active ingredients and excipients, wherein the active ingredients comprise transferrin and a transferrin receptor antibody;
the transferrin and the transferrin receptor antibody have a molar ratio of not less than 1:1.


SEQUENCE LISTING (3 sequences)

SEQ ID NO: 1 - DNA, 22 bp, Artificial Sequence
  Target-2-F
ggggaacttctcctgctagaat

SEQ ID NO: 2 - DNA, 22 bp, Artificial Sequence
  Target-2-R
cagacattttgctctcaagctg

SEQ ID NO: 3 - DNA, 20 bp, Artificial Sequence
  Target-2-P
ttgctgctgcttgacagatt